(19) United States
(12) Patent Application Publication
(10) Pub. No.: US 2023/0263984 A1
(43) Pub. Date: Aug. 24, 2023

(54) Automatic Rescue Breathing Unit with Keying System

(71) Applicants: Daniel A. McCarthy, Tyler, TX (US); Nicolas Ramirez, Rowlett, TX (US); Alvin Hsieh, Plano, TX (US); Anjalin Tomy, Carrollton, TX (US); Randon Johnson, Anna, TX (US); Riyad Ali, Carrollton, TX (US); Alton Gerald Spears, Rowlett, TX (US)

(72) Inventors: Daniel A. McCarthy, Tyler, TX (US); Nicolas Ramirez, Rowlett, TX (US); Alvin Hsieh, Plano, TX (US); Anjalin Tomy, Carrollton, TX (US); Randon Johnson, Anna, TX (US); Riyad Ali, Carrollton, TX (US); Alton Gerald Spears, Rowlett, TX (US)

(21) Appl. No.: 17/932,548

(22) Filed: Sep. 15, 2022

Related U.S. Application Data
(60) Provisional application No. 63/245,093, filed on Sep. 16, 2021.

(51) Int. Cl. A61M 16/08; A61M 16/04; A61M 16/06; A61M 16/10; A61M 16/00; A61H 31/00

(52) U.S. Cl. CPC A61M 16/0816; A61H 31/005; A61M 16/04; A61M 16/024; A61M 16/0605; A61M 16/1005; A61H 2201/107; A61H 2201/5071; A61M 2205/3313; A61M 2205/6081

(57) ABSTRACT

Methods and apparatus for automated detection of an airway device coupled to an automatic rescue breathing unit (ARBU) are disclosed. The automatic detection apparatus includes a keying system that utilizes color adapters coupled to specific airway devices to indicate to a controller that the airway device is a particular size and has a particular airway protection classification (e.g., protected or unprotected). The controller may then determine the proper rescue breath rate and volume (e.g., tidal volume) based on knowing the size and classification of the airway device. In some instances, the controller may also receive information on chest compressions applied to the patient. Automatic detection of the size and classification of the airway device, along with chest compressions, may improve the process of providing automated rescue breaths to a patient.

### PRIORITY CLAIM

This application claims priority to U.S. Provisional Application Serial No. 63/245,093 entitled “Automatic Rescue Breathing Unit with Keying System” filed Sep. 16, 2021, which is incorporated herein by reference in its entirety.

### BACKGROUND

### 1. Field of the Invention

The disclosed embodiments generally relate to a system and method for providing air/oxygen to a subject, and more particularly to providing air/oxygen flow from an air/oxygen supply based on the size and classification of a mask coupled to the air/oxygen supply.

### 2. Description of Related Art

Medical emergencies often call on one or more persons to provide life-saving support. For example, CPR may be performed when a person is not breathing, or breathing inadequately (e.g., during cardiac arrest). CPR generally involves providing air into a person’s lungs via the mouth, or mouth and nose, and performing a series of chest compressions. This may be performed repeatedly to help oxygenate and circulate the blood. Blowing air into the victim’s mouth forces air into the lungs to replace spontaneous respiration and compressing the chest compresses the heart to maintain blood circulation. In a situation in which the heart has stopped beating, performing CPR is intended to maintain a flow of oxygenated blood to the brain and heart, thereby delaying tissue death and extending the opportunity for a successful resuscitation without permanent brain damage. Defibrillation and other advanced life support techniques may also be used to improve the outcome for a victim of cardiac arrest.

CPR techniques can vary depending on the person needing assistance. For example, administering CPR to an adult generally includes providing a set number of full breaths via the mouth, whereas administering CPR to an infant or child may require a larger number of smaller breaths or puffs via the mouth and/or nose. The lower pressure and larger numbers of breaths administered to an infant or child may reduce the likelihood of injury to the respiratory system of the infant or child. Similarly, the force used in administering the chest compressions is reduced when administering CPR to an infant or child. Accordingly, a person who administers CPR must consider several variables and remember a variety of protocols.

CPR is more effective the sooner it is initiated and thus, the time between the onset of the medical emergency and the time of initiating CPR may be critical. Brain cells may begin to die in as little as 4-6 minutes without an adequate supply of oxygen. Unfortunately, medical emergencies can, and often do, happen at locations that are remote to medical facilities and where no trained medical professionals are readily available and, thus, a by-stander may be in the best position to perform CPR.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a representation of an ET tube, according to some embodiments.

FIG. 2 depicts a representation of a CPR mask, according to some embodiments.

FIG. 3 depicts a representation of an ARBU apparatus, according to some embodiments.

FIG. 4 depicts a block diagram of an ARBU apparatus, according to some embodiments

FIG. 5 is an exploded view of an unassembled keying system, according to some embodiments.

FIG. 6 is a cross-sectional representation of an assembled keying system, according to some embodiments.

FIG. 7 depicts a cross-sectional representation of a handpiece and a color sensor, according to some embodiments.

FIG. 8 depicts a representation of color adapters implemented for ET tube airway devices (a) and for CPR mask airway devices (b).

FIG. 9 depicts a flowchart of a rescue breathing process implemented by an ARBU, according to some embodiments

FIG. 10 illustrates an exemplary computer system that may be implemented with an ARBU.

FIGS. 11A-E include illustrations of a portable ventilator system (e.g., ARBU) in accordance with embodiments of the present disclosure.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF EMBODIMENTS

When sudden cardiac arrest (SCA) occurs, the heart stops beating. The loss of heart function causes oxygen saturation, the amount of usable oxygen in the blood, to be depleted. Cardiopulmonary resuscitation (CPR) on the SCA victim is required immediately. CPR consists of delivering chest compressions and air/oxygen (rescue breaths) to the patient. The goal of CPR is for the return of spontaneous circulation of blood to the body. Developments have been made in automatic rescue breathing units (ARBUs) in the treatment of patients suffering SCA to increase the changes of survival. Even with the implementation of ARBUs, however, errors can be made, even by trained professionals, that may decrease the changes of survival. Errors may, for instance, be made in the rates and volumes of rescue breaths provided to the patient due to the complexities involved in determining the rates and volumes.

The present disclosure contemplates various embodiments of an automatic rescue breathing unit (ARBU) that automatically determines the rates and volumes of rescue breaths provided to the patient based on the size and classification of a mask being used to provide air/oxygen to the patient. Automatically determining the rates and volumes of rescue breaths simplifies the process of using the ARBU and increases the changes of patient survival.

Additionally, many current ARBUs provide feedback through a monitor or display that requires the user (e.g., the first responder) to look away from the patient to find out the quality of the compressions being given to the patient. The present disclosure contemplates embodiments where compressions are monitored by a sensor and automatic feedback is provided based on the sensor readings.

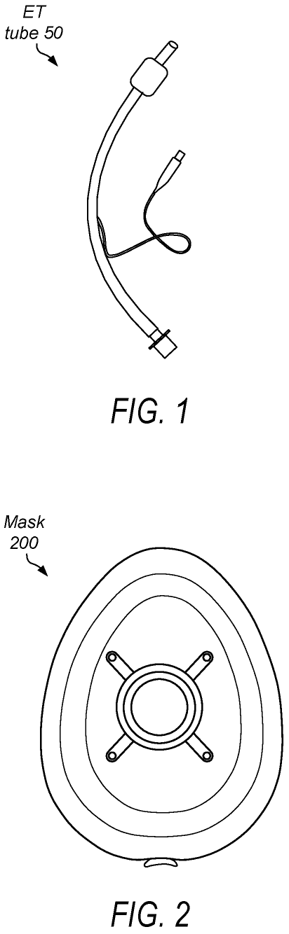

Airway devices used for CPR rescue breaths generally fall under two classifications: protected and unprotected. Protected airway devices keep the patient’s airway open and free of obstructions, such as their tongue. In various embodiments, the protected airway devices used with an ARBU are various sizes of endotracheal (ET) tubes. FIG. 1 depicts a representation of an ET tube, according to some embodiments. In the illustrated embodiment, ET tube 50 is a flexible plastic tube that is inserted into a patient’s airway by a qualified medical professional. When using a protected airway device, rescue breaths can be delivered while compressions are taking place.

Conversely, unprotected airway devices do not keep the patient’s airway open. Accordingly, the airway must be manually opened (e.g., through a head tilt-chin-lift maneuver) and maintained by a CPR responder when giving rescue breaths. Consequently, rescue breaths cannot be given while compressions are taking place. In various embodiments, unprotected airway devices used with the ARBU include CPR masks, which typically come in three different sizes: infant, child, and adult. FIG. 2 depicts a representation of a CPR mask, according to some embodiments. In the illustrated embodiment, mask 200 includes opening 202 for coupling of a air/oxygen tube to the mask and outer contour 202 that is sized and shaped to fit on a face of the patient.

There are typically two common problems that CPR responders face regarding rescue breaths. A first problem is that each airway device has different rescue breath parameters depending on the protected/unprotected classification and the size of the patient. Medical emergencies are inherently high intensity and stressful situations and, in the heat of the moment, it is difficult to impossible for CPR responders to keep the differing parameters in mind and deliver perfect rescue breaths to the patient on a consistent basis.

A second problem is the limitations of common rescue breath methods. The American Heart Association (AHA) recommends that rescue breaths be delivered with the highest oxygen concentration possible. However, it is difficult to meet this recommendation using the most common rescue breath methods. Mouth-to-mouth rescue breaths provide only 17% oxygen. Bag valve masks (BVM) are capable of providing 100% oxygen but are difficult to use.

An ARBU solves these two problems by automating the delivery of rescue breaths. In various embodiments, the ARBU system includes an oxygen tank, ventilator, a keying system, and compression pad. In certain embodiments, the keying system automatically identifies the classification and size of the airway device connected to the ventilator and being used in the CPR procedure. The ventilator may then regulate the rate and volume of rescue breaths delivered to the patient based on the airway device identified by the keying system. In some embodiments, the rate and volume of rescue breaths are regulated per AHA guidelines in combination with the identified airway device. In some embodiments, a compression pad with a sensor detects when chest compressions are being given for the ventilator to determine if the oxygen distribution is continued or ceased.

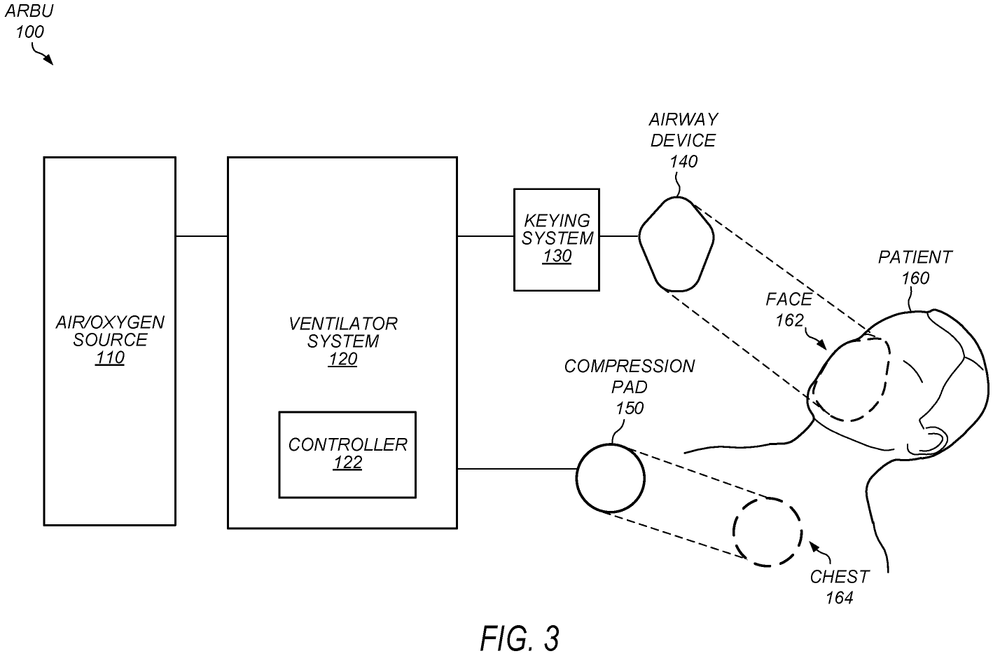

FIG. 3 depicts a representation of an ARBU apparatus, according to some embodiments. In the illustrated embodiment, ARBU 100 includes air/oxygen source 110, ventilator system 120, keying system 130, airway device 140, and compression pad 150. ARBU 100 may be implemented in the treatment of patient 160, shown with face 162 and chest 164. In various embodiments, ARBU 100 is a portable system that may be transported to a location of a patient. Examples of portable ARBU apparatus are described herein.

In various embodiments, source 110 is a source capable of providing pressurized air/oxygen for use by the ventilator system 120. As used herein “pressurized air/oxygen” refers to air/oxygen having a pressure that promotes the flow of the air/oxygen from the ventilator system 120 into the lungs of the patient 160. In some embodiments, pressurized air/oxygen may have a pressure that is above a minimum-pressure threshold, such as 20 centimeters of water (cmH₂O) above ambient air pressure.

In certain embodiments, source 110 is a cylinder containing the pressurized air/oxygen. The pressure of the air/oxygen may be set significantly above the minimum-pressure threshold such that the air/oxygen in the cylinder is maintained above the minimum-pressure threshold as the air/oxygen is expelled from the cylinder and the pressure of the air/oxygen in the source 110 drops as function of the air/oxygen expelled from the cylinder. The source 110 may include a mechanical device, such as a compressor, configured to move and/or pressurize the air/oxygen. Such a mechanical device may be used to pressurize and/or fill a cylinder of the source 110. In one embodiment, the source 110 may include the mechanical device to move the air from the cylinder to the subject.

In some embodiments, ventilator system 120 may receive air/oxygen from the surrounding atmosphere, another source (e.g., an oxygen separation, chemically generated oxygen, or enrichment device), or an air/gas cylinder. Ventilator system 120 may, for example, compress the air/oxygen to a pressure above the minimum-pressure threshold before expelling it through airway device 140.

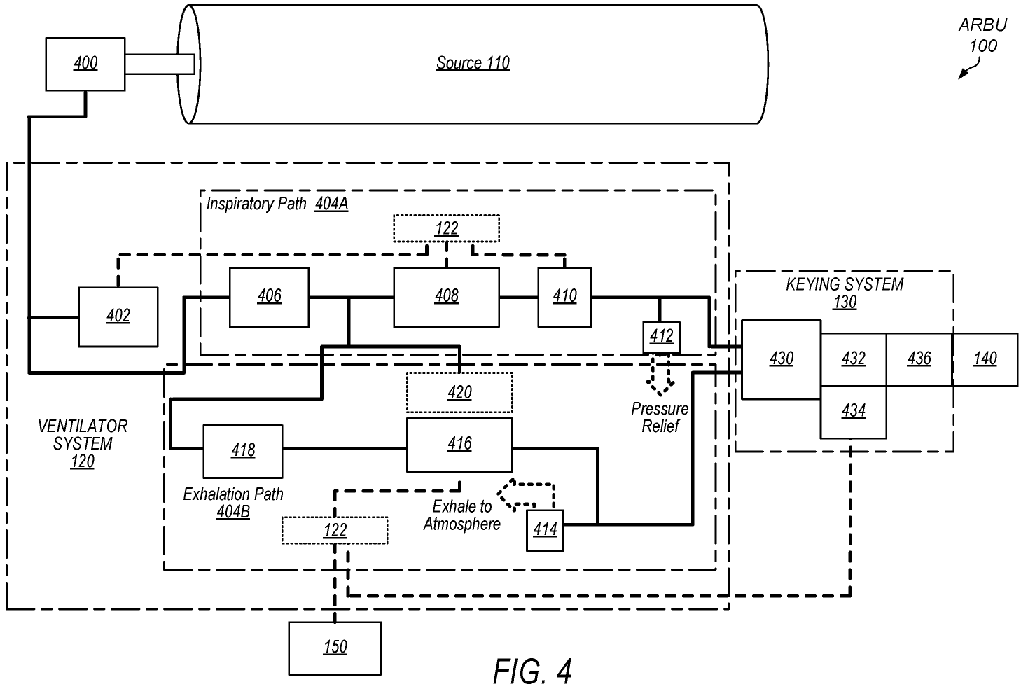

FIG. 4 depicts a block diagram of ARBU 100, according to some embodiments. In the illustrated embodiment, solid lines represent the flow of air/oxygen and dashed lines represent electrical connections. In various embodiments, ventilator system 120 is coupled to source 110 by regulator 400. Regulator 400 may be, for example, a diaphragm regulator used to reduce the pressure of the flow of air/oxygen from source 110. For example, in one embodiment, regulator 400 reduces the pressure to 50 psi. Regulator 400 is coupled to pressure switch 402 in ventilator system 120, which is electrically coupled to controller 122 (note that controller 122 is split into two controllers 122 in the depiction for simplicity in the drawing). When ARBU 100 is in use and there is oxygen in the system, pressure switch 402 is responsible for closing the circuit and turning on the power source for the system. This is necessary to ensure the battery is not being drained when ARBU 100 is not in use. In certain embodiments, ARBU 100 is powered by a battery (e.g., a rechargeable lithium battery) though other portable power sources may be contemplated. Additionally, embodiments may be contemplated where ARBU 100 is capable of being connected to an external power supply such as an electrical outlet or car battery.

After pressure switch 402, the flow path in ventilator system 120 feeds into pressure regulator 406, after which the flow is split into dual limb flow paths - inspiratory path 404A and exhalation path 404B. Inspiratory path 404A provides air/oxygen flow to the patient through airway device 140 coupled by keying system 130 while exhalation path 404B provides a path for monitoring and exhaust of exhalation by the patient.

Inspiratory path 404A includes pressure regulator 406, proportional solenoid valve 408, and flow sensor 410 coupled to pressure switch 402. Proportional solenoid valve 418 and flow sensor 420 are both electrically coupled to controller 122 for monitoring and control of the flow of air/oxygen out of ventilator system 120. In inspiratory path 404A, the pressure of air/oxygen is further reduced through pressure regulator 406 (e.g., from 50 psi to 17 psi) and then flows through proportional solenoid valve 408. Proportional solenoid valve 408 may be controlled by controller 122 (e.g., via a PWM signal) while flow sensor 410 monitors the flow rate from inspiratory path 404A such that controller can adjust the signal to proportional solenoid valve 408 as needed. Backup pressure relief valve 412 along with a check valve and air filter (not shown) may be implemented to protect the patient’s lungs from overpressure and/or particles.

Exhalation path 404B, in combination with inspiratory path 404A, provides a closed loop control for ventilator system 120. Exhalation path 404B includes exhaust valve 414, solenoid valve 416, and pressure regulator 418. It should be noted that pressure regulator 406 may operate to control pressure going into exhalation path 404B, as shown in FIG. 4. Alternatively, exhalation path 404B may include its own pressure regulator with the split in paths coming before pressure regulator 406. Solenoid valve 416 may be, for example, a 3-way solenoid valve controlled by controller 122. Pressure sensor 420 may also be implemented in exhalation path 404B to determine the pressure of air/oxygen returning to ventilator system 120. Pressure sensor 420 may be monitored by controller 122.

The exhalation and pressure relief of ventilator system 120 is controlled in expiratory path 404B. For instance, expired oxygen from the patient flows through exhaust valve 414. Exhaust valve 414 may be pneumatically controlled through solenoid valve 416 and pressure regulator 418. During inhalation, the exhale valve 414 is kept closed unless the pressure reaches unsafe levels. The exhale valve 414 is opened during exhalation allowing expired oxygen to be expelled into the atmosphere.

In various embodiments, compression pad 150 is coupled to controller 122. Compression pad, as shown in FIG. 3 and described herein, may be positioned on chest 164 of patient 160. Compression pad may monitor chest compressions (either automatic or manual) of the patient. Compression pad 150 may have a size and materials selected to be comfortably placed on chest 164 of patient 160. For instance, in one embodiment, compression pad 150 has a diameter of three inches and thickness of a quarter of an inch, and the pad will be made out of platinum cure silicone rubber compound.

Compression pad 160 may include a compression sensor inside the pad. In one embodiment, the sensor is a square FSR sensor. When more pressure is applied to the sensing (square) area of FSR, the resistance is lowered. The sensor detects any force applied on any part of the square surface area of the sensor. In certain embodiments, the sensor is used to detect when compressions are made over compression pad 150 during CPR based on a predetermined pressure. For instance, when the pressure applied is above the predetermined pressure, a compression is detected whereas when the pressure is below the predetermined pressure, no compression is detected. The compression pad 150 may have a relatively small size such that the pad can be used on different sized patients (e.g., adults, children, and infants). In one example embodiments, compression pad 150 is printed using 3D printing and the material is Polylactic acid (PLA). The printed pad may be a master key when creating a mold using silicone rubber compound. Once the mold is created, half of the mold can be filled up with the same compound and left to cure and dry. The next step would be to use a rubber polymer adhesive to place the sensor on the dried portion of the compound and then add the remaining compound to fully fill the mold. Once the compression pad 150 is made from the silicone rubber compound, the resting force on the sensor that is inside the pad will be set to zero to better detect when there is force getting applied based on the pressure being above or below a predetermined pressure.

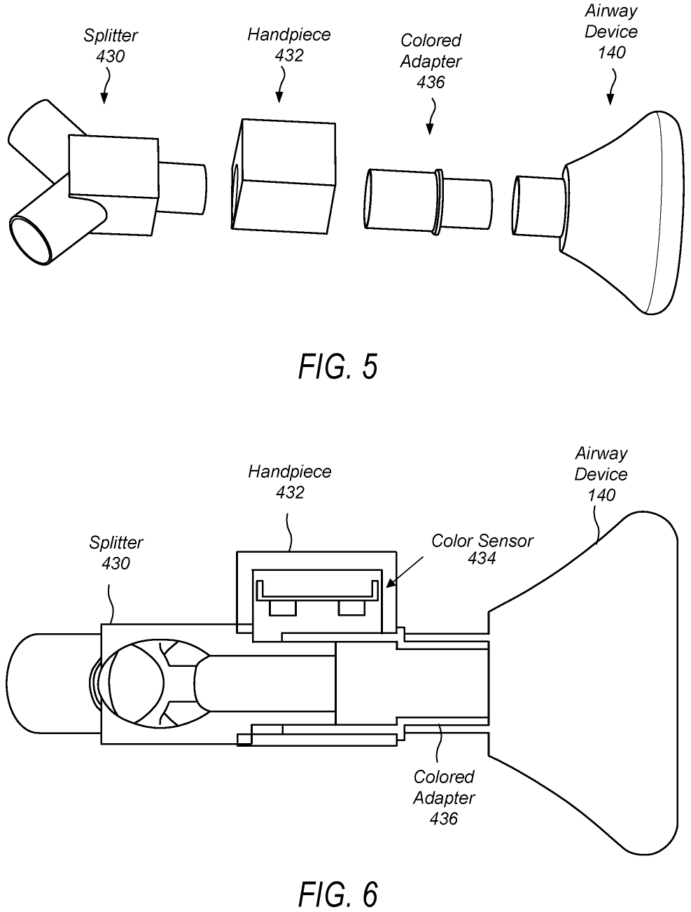

In certain embodiments, as shown in FIGS. 3 and 4, keying system 130 is coupled between ventilator system 120 and airway device 140. FIG. 4 illustrates the disclosed components of keying system 130 in block diagram form. FIG. 5 is an exploded view of unassembled keying system 130, according to some embodiments. FIG. 6 is a cross-sectional representation of assembled keying system 130, according to some embodiments. As shown in FIGS. 4-6, keying system 130 includes splitter 430, handpiece 432, color sensor 434, and colored adapter 436, which is coupled to airway device 140.

Splitter 430 may be, for example, a Y-splitter that connects to the dual limb breathing circuit of ventilator system 120. Thus, splitter 430 connects airway device 140 to both inspiratory path 404A and exhalation path 404B. Splitter 430 is coupled to handpiece 432. Color sensor 434, as shown in FIG. 6, is positioned inside handpiece 432. As further shown in FIG. 6, colored adapter 436 couples to splitter 430 inside handpiece 432. When splitter 430 and colored adapter 436 are coupled, color sensor 434 is positioned to detect the presence and color of the colored adapter.

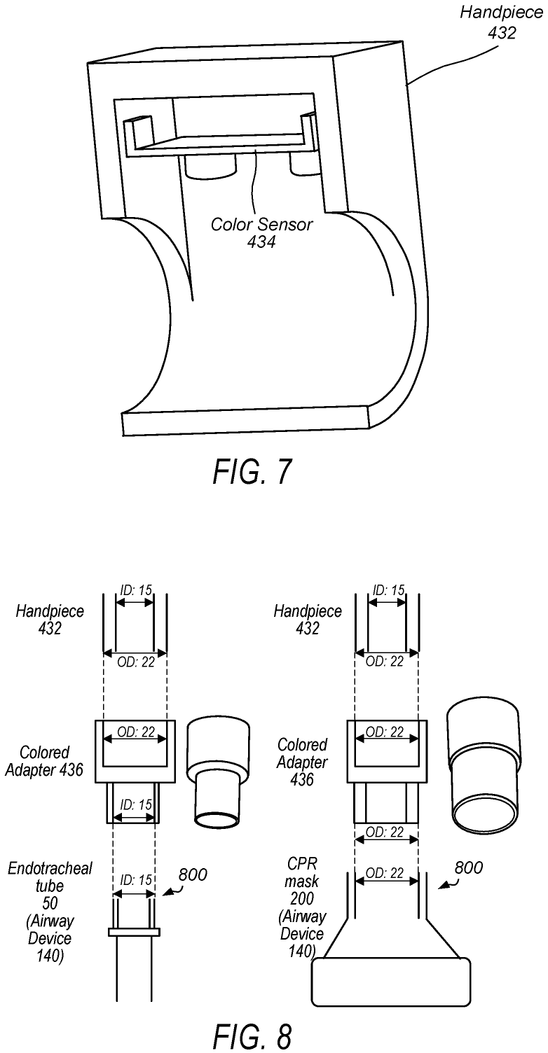

FIG. 7 depicts a cross-sectional representation of handpiece 432 and color sensor 434, according to some embodiments. Handpiece 432 may include a ring with a rectangular compartment at the top to hold color sensor 434. In one embodiments, the inner diameter of handpiece 432 is 27 millimeters, and the length is 42 millimeters. In various embodiments, both handpiece 432 is 3D printed from plastic that is moisture and heat resistant. Color sensor 434 may be coated with a protective epoxy resin. Coating color sensor 434 may inhibit color readings from being interfered with if handpiece 432 is exposed to water, mud, or sand. In some embodiments, a cleaning mechanism using an O-ring and wiper may be added at the opening of handpiece 432 where airway device 140 is attached to prevent any mud or sand from interfering the color reading.

Color sensor 434 may be, for example, a TCS3200 color sensor module, which is widely available in the industry. Color sensor 434 may read colors in terms of the intensities of the red, green, and blue components in an object. Color sensor 434 is based on programmable color and has a high-resolution conversion of light intensity to frequency. The red, green, and blue output frequencies of each color can be read and displayed using widely available software (such as open-sourced Arduino), and then the range of the red, green, and blue frequencies of the seven colors can be set accordingly. Color sensor 434 may be housed inside a compartment in handpiece 432 to control position and interference from external light.

Color adapter 436 is specific to the type and classification of airway device 140 being coupled to ventilator system 120. As described above, airway devices 140 may come in a variety of sizes and classification types for airway protection. Accordingly, in various embodiments, a color may be selected to indicate a specific combination of size of airway device and classification type for airway protection. In certain embodiments, there are seven different airway devices 140 used with ARBU 100 - three different sized ET tubes, three different sized CPR masks, and a venturi mask for continuous oxygen (used when patient is stabilized). Each of these different airway devices has different rescue breath rate and tidal volume requirements. The rescue breath rate and tidal volume requirements for each airway device may also depend on the compressions detected by compression pad 150. Table I below gives examples of rescue breath and tidal volume requirements dependent on compressions for various airway devices.

TABLE I

Example Rescue Breath Regulation Requirements
Adult CPR mask - unprotected airway
Rescue breath tidal volume must be between 540-660 mL.
Rescue breaths are given only when compression pad detects compressions have ceased.
Rescue breaths given at a rate of 10 breaths/minute. Positive pressure rescue breath given over 1 second followed by 5 seconds of passive exhalation.

Child CPR mask - unprotected airway
Rescue breath tidal volume must be between 255-345 mL.
Rescue breaths are given only when compression pad detects compressions have ceased.
Rescue breaths given at a rate of 30 breaths/minute. Positive pressure rescue breath given over 1 second followed by 1 second of passive exhalation.

Infant CPR mask - unprotected airway
Rescue breath tidal volume must be between 80-120 mL.
Rescue breaths are given only when compression pad detects compressions have ceased.
Rescue breaths given at a rate of 30 breaths/minute. Positive pressure rescue breath given over 1 second followed by 1 second of passive exhalation.

Adult ET tube - protected airway
Rescue breath tidal volume must be between 540-660 mL.
Rescue breaths are given continuously and do not pause during compressions.
Rescue breaths given at a rate of 10 breaths/minute. Positive pressure rescue breath given over 1 second followed by 5 seconds of passive exhalation.

Child ET Tube - protected airway
Rescue breath tidal volume must be between 255-345 mL.
Rescue breaths given continuously and do not pause during compressions.
Rescue breaths given at a rate of 30 breaths/minute. Positive pressure rescue breath given over 1 second followed by 1 second of passive exhalation.

Infant ET Tube - protected airway
Rescue breath tidal volume must be between 80-120 mL.
Rescue breaths given continuously and do not pause during compressions.
Rescue breaths given at a rate of 30 breaths/minute. Positive pressure rescue breath given over 1 second followed by 1 second of passive exhalation.

Continuous flow oxygen mask
Continuous flow rate: 10-15 L/min

In certain embodiments, a color adapter 436 with a specific color may be assigned to each of the different airway devices used with ARBU 100. For example, each of the airway devices in Table I has its own specified color applied to it and this specified color is then applied to a color adapter 436 suppled with the airway device. The color adapter 436 with the applied color may be attached to its corresponding airway device 140 or have some other means for ensuring the specific color adapter is used with its specific airway device.

In various embodiments, color adapters 436 are connectors that adapt the connection point on airway device 140 to the dimensions of handpiece 432. For example, ET tube airway devices and CPR masks have different connection dimensions. Thus, color adapter 436 (in addition to indicating color for detection) adapts the connection dimensions of its corresponding airway device 140 to the connection dimensions of handpiece 432. FIG. 8 depicts a representation of color adapters 436 implemented for ET tube airway devices (a) and for CPR mask airway devices (b). As shown in FIG. 8, both airway devices have connection dimensions 800 that are adapted to the connection dimensions of handpiece 432 by color adapters 436.

In some embodiments, color is added to color adapters 436 using colored electrical tape. For example, colored electrical tape may be added to the end of color adapters 436 intended to connect to splitter 430 such that the tape is positioned inside handpiece 432 and detected by color sensor 434. In some embodiments, a clear coat sealant may be placed over the tape to prevent damage to the tape during use. Other embodiments may be contemplated for providing color to color adapters 436. For example, color adapters 436 may be manufactured out of material having the specified color needed, the adapters may be painted with the specified color, etc.

In certain embodiments, as described herein, keying system 130 may determine the size and classification of airway device 140 coupled to ventilator system 120 according to the color of colored adapter 436 detected by color sensor 434. From the detected size and classification of airway device 140, ventilator system 120 (e.g., controller 122) may then determine a rate and volume (e.g., tidal volume) of rescue breaths provided to patient 160, shown in FIG. 1. In some embodiments, the rate and volume of rescue breaths may be further controlled according to chest compressions detected by compression pad 150.

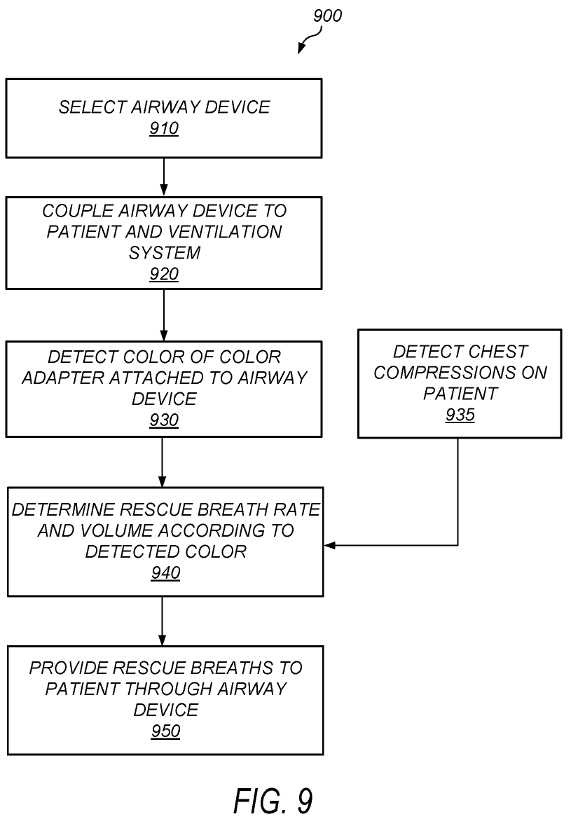

FIG. 9 depicts a flowchart of a rescue breathing process implemented by ARBU 100, according to some embodiments. In rescue breathing process 900, a user (such as a first responder or other person providing treatment) selects the correct airway device in 910. The airway device may be selected based on the size and status of the patient. The airway device may be selected based on the patient being an adult, a child, or infant to determine size and an ET tube or a CPR mask (or a constant oxygen flow mask) based on the status of the patient (e.g., whether the patient needs to be intubated with the ET tube or only needs a CPR mask).

After selection of the airway device, in 920, the airway device may be coupled to the patient (e.g., placed on face 162 of patient 160) and coupled to ventilator system 120 using keying system 130. As discussed above, when the user selects the proper airway device, the airway device 140 has a corresponding color adapter 436 used to connect the airway device to the ventilator system 120 through keying system 130. Further, as described above, the color adapter 436 has a color that indicates the size and airway protection classification of the airway device 140.

In 930, ventilator system 120 may detect a color of color adapter 436 attached to airway device 140. As described above, the color may be detected by color sensor 434, which is electrically coupled to controller 122 in ventilator system 120, as shown in FIG. 4. In 940, ventilator system 120 (by controller 122) may determine a rate and volume (e.g., tidal volume) of rescue breaths to be applied to the patient through airway device 140. In 950, the rescue breaths may then be provided to the patient through dual limb breathing circuit (e.g., inspiratory path 404A and exhalation path 404B) in ventilator system 120 with the determined rate and volume. As described above, the dual limb breathing circuit allows for closed loop control of the rate and volume of rescue breaths provided to the patient to ensure the proper rate and volumes are being provided.

In some embodiments, process 900 includes detecting chest compressions in 935. For example, chest compressions can be detected (e.g., detection of presence or rate) by compression pad 150 placed on chest 164 of patient 140. The detected compressions may then be used in the determination of rescue breath rate and volume in 940. As discussed above and shown in Table I, rescue breaths may be stopped or adjusted based on the detection of chest compressions.

### Example Computer System

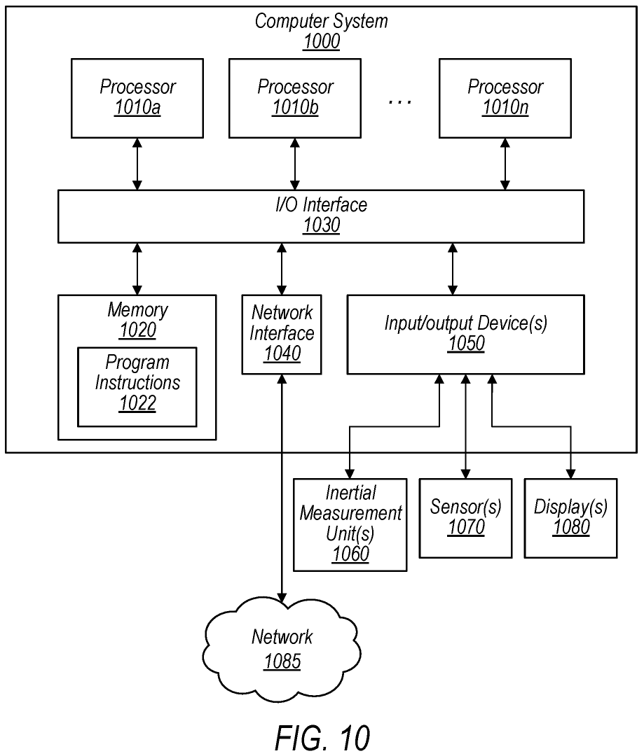

FIG. 10 illustrates exemplary computer system 1000 that may be implemented with an ARBU. In different embodiments, computer system 1000 may be any of various types of devices, including, but not limited to, a personal computer system, desktop computer, laptop, notebook, tablet, slate, pad, or netbook computer, handheld computer, workstation, network computer, a camera, a set top box, a mobile device, a consumer device, video game console, handheld video game device, application server, storage device, a television, a video recording device, a peripheral device such as a switch, modem, router, or in general any type of computing or electronic device.

Various embodiments of program instructions for controlling ARBU, for example, by controller 122 as described herein, may be executed in one or more computer systems 1000, which may interact with various other devices. Note that any component, action, or functionality described above with respect to FIGS. 1-9 may be implemented on one or more computers configured as computer system 1000 of FIG. 10, according to various embodiments. In the illustrated embodiment, computer system 1000 includes one or more processors 1010 coupled to a system memory 1020 via an input/output (I/O) interface 1030. Computer system 1000 further includes a network interface 1040 coupled to I/O interface 1030, and one or more input/output devices 1050, such as cursor control device 1060, keyboard 1070, and display(s) 1080. In some cases, it is contemplated that embodiments may be implemented using a single instance of computer system 1000, while in other embodiments multiple such computer systems, or multiple nodes making up computer system 1000, may be configured to host different portions or instances program instructions for re-mapping, rendering, encoding, or decoding points cloud as described above for various embodiments. For example, in one embodiment some elements of the program instructions may be implemented via one or more nodes of computer system 1000 that are distinct from those nodes implementing other elements.

In some embodiments, computer system 1000 may be implemented as a system on a chip (SoC). For example, in some embodiments, processors 1010, memory 1020, I/O interface 1030 (e.g., a fabric), etc. may be implemented in a single SoC comprising multiple components integrated into a single chip. For example, an SoC may include multiple CPU cores, a multi-core GPU, a multi-core neural engine, cache, one or more memories, etc. integrated into a single chip. In some embodiments, an SoC embodiment may implement a reduced instruction set computing (RISC) architecture, or any other suitable architecture.

System memory 1020 may be configured to store compression or decompression program instructions 1022 and/or sensor data accessible by processor 1010. In various embodiments, system memory 1020 may be implemented using any suitable memory technology, such as static random-access memory (SRAM), synchronous dynamic RAM (SDRAM), nonvolatile/Flash-type memory, or any other type of memory. In the illustrated embodiment, program instructions 1022 may be configured to implement any of the functionality described above. In some embodiments, program instructions and/or data may be received, sent or stored upon different types of computer-accessible media or on similar media separate from system memory 1020 or computer system 1000.

In one embodiment, I/O interface 1030 may be configured to coordinate I/O traffic between processor 1010, system memory 1020, and any peripheral devices in the device, including network interface 1040 or other peripheral interfaces, such as input/output devices 1050. In some embodiments, I/O interface 1030 may perform any necessary protocol, timing or other data transformations to convert data signals from one component (e.g., system memory 1020) into a format suitable for use by another component (e.g., processor 1010). In some embodiments, I/O interface 1030 may include support for devices attached through various types of peripheral buses, such as a variant of the Peripheral Component Interconnect (PCI) bus standard or the Universal Serial Bus (USB) standard, for example. In some embodiments, the function of I/O interface 1030 may be split into two or more separate components, such as a north bridge and a south bridge, for example. Also, in some embodiments some or all of the functionality of I/O interface 1030, such as an interface to system memory 1020, may be incorporated directly into processor 1010.

Network interface 1040 may be configured to allow data to be exchanged between computer system 1000 and other devices attached to a network 1085 (e.g., carrier or agent devices) or between nodes of computer system 1000. Network 1085 may in various embodiments include one or more networks including but not limited to Local Area Networks (LANs) (e.g., an Ethernet or corporate network), Wide Area Networks (WANs) (e.g., the Internet), wireless data networks, some other electronic data network, or some combination thereof. In various embodiments, network interface 1040 may support communication via wired or wireless general data networks, such as any suitable type of Ethernet network, for example; via telecommunications/telephony networks such as analog voice networks or digital fiber communications networks; via storage area networks such as Fibre Channel SANs, or via any other suitable type of network and/or protocol.

Input/output devices 1050 may, in some embodiments, include one or more display terminals, keyboards, keypads, touchpads, scanning devices, voice or optical recognition devices, or any other devices suitable for entering or accessing data by one or more computer systems 1000. Multiple input/output devices 1050 may be present in computer system 1000 or may be distributed on various nodes of computer system 1000. In some embodiments, similar input/output devices may be separate from computer system 1000 and may interact with one or more nodes of computer system 1000 through a wired or wireless connection, such as over network interface 1040.

As shown in FIG. 10, memory 1020 may include program instructions 1022, which may be processor-executable to implement any element or action described above. In one embodiment, the program instructions may implement the methods described above. In other embodiments, different elements and data may be included.

Computer system 1000 may also be connected to other devices that are not illustrated, or instead may operate as a stand-alone system. In addition, the functionality provided by the illustrated components may in some embodiments be combined in fewer components or distributed in additional components. Similarly, in some embodiments, the functionality of some of the illustrated components may not be provided and/or other additional functionality may be available.

Those skilled in the art will also appreciate that, while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 1000 may be transmitted to computer system 1000 via transmission media or signals such as electrical, electromagnetic, or digital signals, conveyed via a communication medium such as a network and/or a wireless link. Various embodiments may further include receiving, sending or storing instructions and/or data implemented in accordance with the foregoing description upon a computer-accessible medium. Generally speaking, a computer-accessible medium may include a non-transitory, computer-readable storage medium or memory medium such as magnetic or optical media, e.g., disk or DVD/CD-ROM, volatile or non-volatile media such as RAM (e.g., SDRAM, DDR, RDRAM, SRAM, etc.), ROM, etc. In some embodiments, a computer-accessible medium may include transmission media or signals such as electrical, electromagnetic, or digital signals, conveyed via a communication medium such as network and/or a wireless link.

### Example ARBU Apparatus

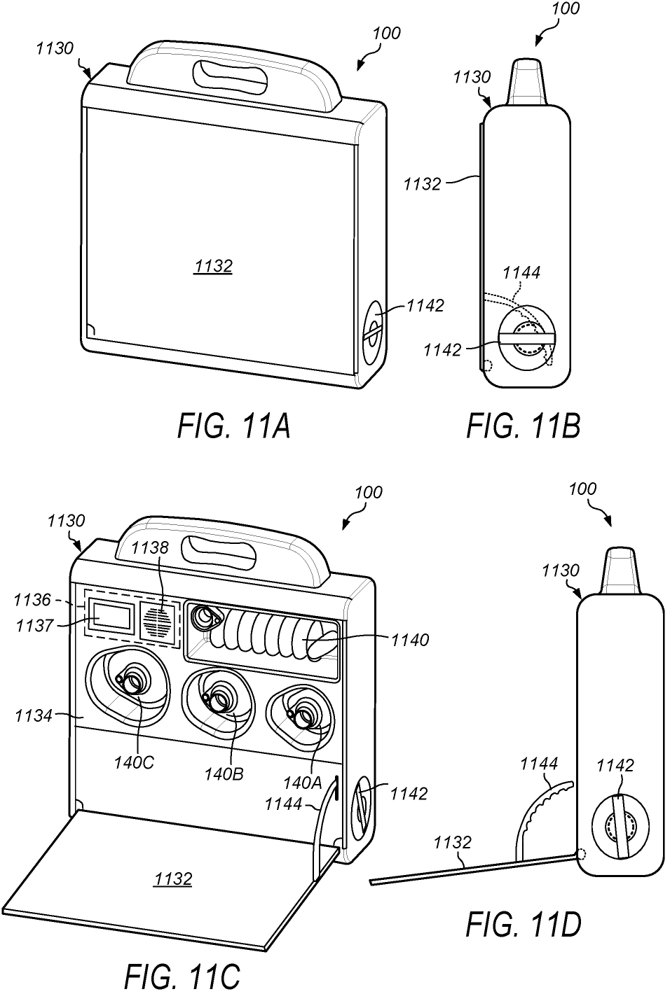

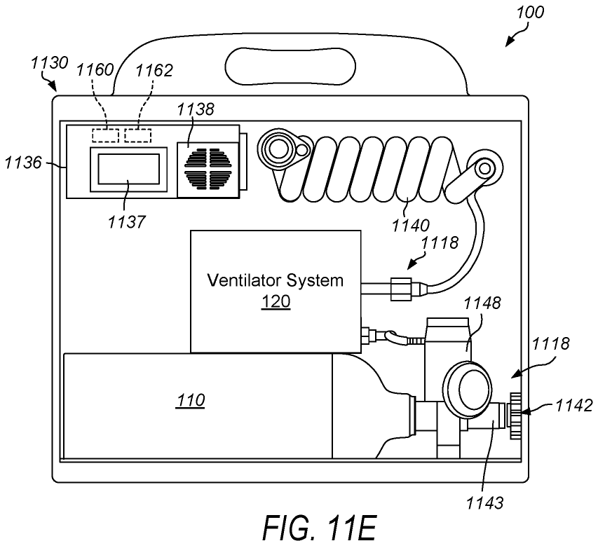

FIGS. 11A-11E include illustrations of a portable ventilator system (e.g., ARBU 100) in accordance with embodiments of the present disclosure. In the illustrated embodiments, ARBU 100 includes an enclosure 1130 having a lid 1132, an internal cover 1134, airway devices 140A, 140B, 140C (and additional airway devices such ET tubes, not shown) provided in respective cradles of the internal cover 1134, a communications device 1136 including a display 1137 and a microphone/speaker 1138, a flexible respirator hose/conduit 1140, a case plug 1142, and a ratchet arm 1144. As illustrated in FIG. 11E having internal cover 1134 removed, portable ARBU 100 also includes, internally, an air/oxygen source cylinder 110, an air regulation system 1118 including a regulator 1148, and a ventilator system 120.

Enclosure 1130 may include a case that protects and organizes the components of the portable ARBU 100. For example, the enclosure may include a plastic or metal case that is of suitable size to be readily transported by a user, such as a “Pelican” case manufactured by Pelican-Case, having headquarters in San Antonio, Texas. In some embodiments, opening of the enclosure 1130 may provide activation of one or more functions. For example, opening of the lid 1132 may cause the ratchet arm 1144 to engage and rotate the case plug 1142, which is coupled to valve 1143, thereby rotating a valve of the air/oxygen source into an opened/activated position that facilitates the delivery of air/oxygen from the source 16.

Further, it is anticipated that dialing a 911 operator may be helpful, thus, certain embodiments may include a telephonic device, such as a cell phone, or other communications device, that may enable a user to make a call to 911 in conjunction with use of the ARBU 100. For example, in one embodiment, a button, voice activated, or user activated switch may be located on the ARBU 100 such that a user may place a call to a 911 operator. In one embodiment, the ARBU 100 may be configured to only place calls to 911, and not receive 911 calls. Accordingly, there may be no additional access fee required as connections to 911 may not be charged a fee and operation of such a unidirectional cellular may not require a monthly access fee. One embodiment may include the ARBU 100 configured to provide for wireless internet access, satellite phone access, access to an operator, access to an emergency room physician, access to a CPR support center, or the like.

In some embodiments the (portable) ARBU 100 includes a communications device that, during use, automatically contacts an emergency response entity in response to activation of the ARBU 100. An emergency response entity may include paramedics, a fire department, a police department, local security personnel, local medical personnel (e.g., a nurse station), or the like. In some embodiments, automatically contacting an emergency response entity comprises notifying an emergency responder of activation of the portable ventilator system. For example, the communication device 1136 of ARBU 100 may place a call to an emergency responder (e.g., call “911”), may send a textual message to an emergency responder, or a similar alert. In some embodiments, activation of the portable ventilator system includes removing the ARBU 100 from a storage location (e.g., removing the system from a case, or rack), opening the enclosure 1130 of the ARBU 100, coupling of a ventilator airway device to an air/oxygen source and/or an air/oxygen regulator system of the ARBU 100, initiation of air/oxygen flow through at least a portion of the ARBU 100, manual activation of an input of the ARBU 100 by a user (e.g., pressing a power/start/alert/activation button or initiating air/oxygen flow from source 1116), or the like. In some embodiments, the communications device 1136 may be manually activated by a user to communicate with an emergency response entity. In some embodiments, communications device 1136 may be activated automatically and/or manually. For example, the communications device 1136 may initiate contact of an emergency response entity automatically upon activation and/or in response to a user manually requesting that such a contact/communication be established.

In some embodiments, the communication device 1136 may be used to communicate instructions to a user. In one embodiment, audible instructions may be provide to the user via speaker 1138. For example, after contacting an emergency response entity, a representative (e.g., a 911 dispatcher) may provide instructions describing how to provide CPR to the subject using the ARBU 100. In some embodiments, pre-stored audible instructions may be provided to the user via the speaker 1138 upon activation of the ARBU 100 and/or a manual request from a user for instructions (e.g., pressing a button to request the audible instructions). In one embodiment, visual instructions may be provided to a user via the display 1137. For example, upon activation of the ARBU 100, textual, graphical, and/or animated instructions may be displayed to the user via the display 1137. This may include activating lights (e.g., LEDs) or a displayed textual description to indicate the status of the ARBU 100 (e.g., active, functioning properly, not functioning properly, operating in infant/child/adult mode), what airway device to choose, how to couple the airway device to the subject, etc. In some embodiments, graphical animations of how to use the ARBU 100 may be provided via the display 1137.

In some embodiments, the communications device 1136 may provide for two-way communication between the emergency response entity and a user of the portable ventilator system. For example, a user may provide input (e.g., press a button or speak into the microphone 1138) and may receive feedback from an emergency responder (e.g., audible instructions via the speaker 1138 or textual/graphical instructions via the display 1137).

In certain embodiments, ARBU 100 may include a processor 1160 (a controller) configured to implement certain routines to provide various functions described herein. In some embodiments, a ventilator system 110 may include a non-transitory computer readable storage medium 1162 (e.g., a floppy disk, random access memory (RAM), read only memory (ROM), hard drive, flash memory, or the like) having program instruction stored thereon. The program instructions may be executable by a processor to implement various functions described herein. For example, the program instructions may be executed to provide displayed or audible instructions for using the ARBU 100, to contact an emergency response entity, or the like.

Although a portable configuration may be advantageous in certain scenarios, the ARBU 100 may also include a generally non-portable configuration. For example, where it is anticipated that the ARBU 100 may be used for an extended period of time (e.g., an hour or more), the source 1116 may be increased in size or supplemented by another supply, such as a large cylinder, a second small cylinder, or stand-alone oxygen supply unit generally available in a hospital or similar medical facility or aircraft, fire truck, ambulance or other emergency vehicle. Such an embodiment may be employed within a health care facility to ensure that flow parameters are selected based on the airway device coupled to the ARBU 100, and ensure that even trained professionals do not inadvertently select inappropriate flow parameters.

Although certain embodiments have been discussed in detail, other embodiments of the ARBU 100 are within the scope of this disclosure. For example, while the current description focuses on a singular ventilation control system with a plurality of preset ventilator parameters which are selected based on the particular airway device in use, another embodiment may include a plurality of ventilation control system/airway device combination units where the airway device and ventilation control system exist as a single unit. In this embodiment, the system would include three or more separate ventilation control system/airway device combinations with the particular airway device corresponding to the airflow characteristics which that particular ventilation control system is preset to deliver. To use this particular embodiment, the user would, instead of selecting the desired airway device and attaching it to the ventilation control system, select from a plurality of ventilation control system/airway device combination units, the airway device size corresponding to the parameters which are preset into the device. Further, the ARBU 100 has been discussed in the context of a ventilator system; however, other embodiments may include similar forms of air/oxygen delivery devices, such as a respirator.

In other embodiments, the ARBU 100 may incorporate and/or be combined with various medical devices. For example, in one embodiment, the ARBU 100 may be provided in conjunction with and/or include a defibrillator. As discussed above, when air/oxygen is delivered via the ventilator supply system, and is used to assist respiration and oxygenation, the defibrillator may, then, be employed in an attempt to correct lethal cardiac electrical activity. For instance, CPR may be performed prior to administering an electric shock to the heart via the defibrillator, as this may increase the likelihood of defibrillation success, and improve the chance for victim survival. The ARBU 100 may include a carbon dioxide detector in one embodiment. For example, a carbon dioxide detector cartridge may be placed in the system and configured to assist in the detection of the presence of carbon dioxide and confirm air movement from the patient. Further, embodiments may include airway devices such as oral airways, nasal trumpets, laryngeal mask airways and CPR prompts (e.g., audible beep to prompt when to do compressions), or Broslow charts. Further, embodiments may include laryngeal mask airway, king airway or other advanced airway device connections having a keying feature similar to those described herein.

The ARBU 100 may include medications provided with the unit that could be administered to the subject. For example, the ARBU 100 may include a compartment configured to contain Epinephrine for allergic reactions or asthmatic emergencies. These could be used only under stringent guidelines, and/or specific direction from one licensed or allowed to prescribe or administer drugs.

In some embodiments, systems and functions as described above are implemented without any electronic components. In certain embodiments, for example, a system implements control of air/oxygen (e.g., inspiration volume, cycle time, etc.), keying, and/or mode of operation by way of non-electronic mechanisms and methods. For example, in one embodiment, the system activates a supplemental oxygen mode by a pneumatic mechanism.

The methods described herein may be implemented in software, hardware, or a combination thereof, in different embodiments. In addition, the order of the blocks of the methods may be changed, and various elements may be added, reordered, combined, omitted, modified, etc. Various modifications and changes may be made as would be obvious to a person skilled in the art having the benefit of this disclosure. The various embodiments described herein are meant to be illustrative and not limiting. Many variations, modifications, additions, and improvements are possible. Accordingly, plural instances may be provided for components described herein as a single instance. Boundaries between various components, operations and data stores are somewhat arbitrary, and particular operations are illustrated in the context of specific illustrative configurations. Other allocations of functionality are envisioned and may fall within the scope of claims that follow. Finally, structures and functionality presented as discrete components in the example configurations may be implemented as a combined structure or component. These and other variations, modifications, additions, and improvements may fall within the scope of embodiments as defined in the claims that follow.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. The words “include”, “including”, and “includes” mean including, but not limited to. As used throughout this application, the singular forms “a”, “an” and “the” include plural referents unless the content clearly indicates otherwise. Thus, for example, reference to “a mask” includes a combination of two or more masks. The term “coupled” means directly or indirectly connected.

What is claimed is:

1. A rescue breath apparatus, comprising:
a set of multiple airway devices, the airway devices being configured to be placed on a face of a human patient, wherein the set includes at least two airway devices having different sizes intended for different size human faces, and wherein the set includes at least two airway devices with different classifications of airway protection;
a set of adapters, individual adapters being coupled to individual airway devices, wherein each adapter is color coded according to the airway device to which the adapter is coupled, and wherein the color code of the adapter indicates a size of the airway device and a classification of airway protection for the airway device;
an air/oxygen source;
a ventilator system coupled to the air/oxygen source, the ventilator system comprising:
a connector configured to couple to the adapter associated with an airway device selected for the patient; and
a handpiece comprising a color sensor, the handpiece configured to be positioned around the connector and the adapter when the connector is coupled to the adapter, wherein the color sensor is configured to determine a color of the adapter coupled to the connector;
wherein the ventilator system is configured to provide air/oxygen to the patient through the selected airway device coupled to the ventilator system,
a controller comprising a computer processor, the controller being configured to determine a rate and volume of rescue breaths provided by the ventilator system based on the size of the coupled airway device and the classification of airway protection for the coupled airway device indicated by the color determined by the color sensor.

2. The apparatus of claim 1, wherein the different sizes of airway devices are intended for human faces selected from the following different sizes of human faces: adult, child, and infant.

3. The apparatus of claim 1, wherein the classification of airway protection is either a protected airway device or an unprotected airway device.

4. The apparatus of claim 3, wherein the protected airway device is an endotracheal (ET) tube.

5. The apparatus of claim 3, wherein the unprotected airway device is a cardiopulmonary resuscitation (CPR) mask.

6. The apparatus of claim 1, further comprising a compression pad configured to be positioned on a chest of the patient, the compression pad having a sensor configured to detect chest compressions being performed on the patient.

7. The apparatus of claim 6, wherein the controller is configured to determine whether air/oxygen is provided to the patient based on chest compressions being detected by the sensor in the compression pad.

8. The apparatus of claim 7, wherein the controller is configured to inhibit air/oxygen being provided to the patient when a chest compression is detected by the sensor and the classification of airway protection for the coupled airway device indicates the airway device is an unprotected airway device.

9. The apparatus of claim 1, wherein the controller is configured to determine a safe pressure limit for the rescue breaths provided by the ventilator system based on the size of the coupled airway device and the classification of airway protection for the coupled airway device indicated by the color determined by the color sensor.

10. The apparatus of claim 1, wherein the controller is configured to determine a rate of positive pressure rescue breaths versus a rate passive exhalation provided by the ventilator system based on the size of the coupled airway device and the classification of airway protection for the coupled airway device indicated by the color determined by the color sensor.

11. The apparatus of claim 1, wherein at least one of the airway devices is a continuous flow oxygen mask, and wherein the controller is configured to provide a continuous flow of oxygen based on the color determined by the color sensor indicating the airway device is the continuous flow oxygen mask.

12. A method, comprising:
detecting, by a color sensor located in a handpiece of a ventilator system, a color of an adapter coupled to a connector inside of the handpiece, wherein the color of the adapter is detected by a color sensor positioned in the handpiece, wherein the adapter is coupled to an airway device selected from a set of multiple airway devices, the airway device being positioned on a face of a human patient, and wherein the set of multiple airway devices includes at least two airway devices having different sizes intended for different size human faces and at least two airway devices with different classifications of airway protection;
determining, by a controller coupled to the ventilator system, a rate and volume of rescue breaths to be provided by the ventilator system based on a size and a classification of airway protection for the airway device coupled to the ventilator system, the size and classification corresponding to the color detected by the color sensor, wherein the controller includes a computer processor; and
providing, by the ventilator system, air/oxygen from an air/oxygen source to the patient through the airway device coupled to the ventilator system, the air/oxygen being provided according to the determined rate and volume of rescue breaths.

13. The method of claim 12, wherein the different sizes of airway devices in the set are intended for human faces selected from the following different sizes of human faces: adult, child, and infant.

14. The method of claim 12, wherein the classification of airway protection is either a protected airway device or an unprotected airway device.

15. The method of claim 12, further comprising detecting, by a sensor positioned in a compression pad placed on a chest of the patient, chest compressions being performed on the patient.

16. The method of claim 15, further comprising determining, by the controller, whether air/oxygen is provided to the patient based on chest compressions being detected by the sensor in the compression pad.

17. The method of claim 16, further comprising inhibiting, by the controller, air/oxygen being provided to the patient when a chest compression is detected by the sensor and the classification of airway protection for the coupled airway device indicates the airway device is an unprotected airway device.

18. The method of claim 12, further comprising determining, by the controller, a safe pressure limit for the rescue breaths provided by the ventilator system based on the size of the coupled airway device and the classification of airway protection for the coupled airway device indicated by the color determined by the color sensor.

19. The method of claim 12, further comprising determining, by the controller, a rate of positive pressure rescue breaths versus a rate passive exhalation provided by the ventilator system based on the size of the coupled airway device and the classification of airway protection for the coupled airway device indicated by the color determined by the color sensor.

20. The method of claim 12, further comprising providing a continuous flow of oxygen based on the color detected by the color sensor corresponding to the airway device being a continuous flow oxygen mask.
